(19) United States
(12) Patent Application Publication
(10) Pub. No.: US 2022/0036427 A1
(43) Pub. Date: Feb. 3, 2022

(54) METHOD FOR MANAGING IMMERSION LEVEL AND ELECTRONIC DEVICE SUPPORTING SAME

(71) Applicant: Dang Chan OH, Jeonju-si Jeollabuk-do (KR)

(72) Inventor: Dang Chan OH, Jeonju-si Jeollabuk-do (KR)

(21) Appl. No.: 17/298,421

(22) PCT Filed: Nov. 20, 2019
(86) PCT No.: PCT/KR2019/015921

§ 371 Date: May 28, 2021

(30) Foreign Application Priority Data
Nov. 30, 2018 (KR) 10-2018-0152520

(51) Int. Cl. G06Q 30/06; G06K 9/00; G06F 3/01

(52) U.S. Cl. CPC G06Q 30/0631; G06F 3/013; G06K 9/00315

(57) ABSTRACT

An electronic device including a display, a camera, and a processor is proposed. The processor may be set to activate the camera while outputting a content-playback screen on the display when a playback of content is requested, obtain a face-related image of a user by using the activated camera, extract eye-blink information, information on a change in outer corners of eyes and information on a change in corners of mouth from the face-related image, and calculate an immersion level of the user with the content on the basis of the eye-blink information, the information on the change in outer corners of eyes, and the information on the change in corners of mouth.

### TECHNICAL FIELD

The present invention relates to managing an immersion level and, more particularly, to a method for managing an immersion level and an electronic device supporting the same, wherein the immersion level for content is detected on the basis of user's face-related information obtained while a user is reading the content, and the detected immersion level is used to support content recommendation.

### BACKGROUND ART

Recently, through the rapid progress of information technology, wired and wireless network infrastructures have been expanded, and various mobile smart devices such as smartphones and tablet PCs have rapidly become popular. Such technological changes have created a new era and cultural content environment by enabling real-time communication and greatly reducing temporal and spatial constraints required for online access.

As the cultural industry that includes interactive digital games and various learning content has increased in recent years, the importance of cognitive elements such as immersion or concentration has emerged.

Meanwhile, in order to determine cognitive elements such as immersion or concentration in the related art, there have been attempts to measure a length of time during which a user displays a specific content on a display and measure a level of immersion in the corresponding content on the basis of that time. However, even when an electronic device is displaying the content on the display, since the electronic device is unable to identify whether the user is viewing the content or is doing something else, the level of immersion measured on the basis of the content display time is very inaccurate, so there is a problem with low reliability.

### DISCLOSURE

### Technical Problem

In order to solve the above-described problem, an objective of the present invention is to provide a method for managing an immersion level and an electronic device supporting the same, wherein an immersion level may be calculated more accurately by complexly analyzing various information obtained from a face of a user who is reading or viewing content.

In addition, another objective of the present invention is to provide the method for managing an immersion level and the electronic device supporting the same, the method supporting functionality in which a user's rating for corresponding content is determined on the basis of the more accurately calculated immersion level, and other content may be recommended on the basis of the determined user's rating.

However, the objectives of the present invention are not limited to the objectives mentioned above, and other objectives not mentioned herein will be clearly understood by those skilled in the art from the following description.

### Technical Solution

An electronic device for achieving the above-described objective includes: a display for outputting content; a camera for collecting a user-related image; and a processor functionally connected to the display and the camera. The processor may be set to activate the camera while outputting a content-playback screen on the display when a playback of the content is requested, obtain a face-related image of a user by using the activated camera, extract eye-blink information, information on a change in outer corners of eyes, and information on a change in corners of mouth from the face-related image, and calculate an immersion level of the user with the content on the basis of the eye-blink information, the information on the change in outer corners of eyes, and the information on the change in corners of mouth.

In particular, the processor may be set to calculate an immersion-level score on the basis of the eye-blink information, generate emotion information of the user on the basis of the information on the change in outer corners of eyes and the information on the change in corners of mouth, and correct the immersion-level score on the basis of the emotion information.

Alternately, the processor may be set to increase the immersion-level score calculated on the basis of the eye-blink information by a specified value when the emotion information indicates an interested emotional state.

Alternately, the processor may be set to lower the immersion-level score calculated on the basis of the eye-blink information by a specified value when the emotion information indicates an expressionless state.

Meanwhile, the electronic device further includes a communication circuit for establishing a communication channel with a service device for providing the content. In this case, the processor may be set to transmit an immersion-level score to the service device through the communication circuit, receive a content recommendation list generated on the basis of the immersion-level score from the service device, and output the received content recommendation list on the display.

A method for managing an immersion level according to an exemplary embodiment of the present invention includes: receiving a playback request of content by a terminal device; activating the camera while outputting a content-playback screen according to the playback request of the content; obtaining a face-related image of a user by using the activated camera; calculating the immersion level of the user for the content on the basis of the face-related image; providing information of the calculated immersion level to a designated service device; and outputting a content recommendation list by receiving the content recommendation list, which is generated on the basis of the information of the immersion level, from the service device.

Here, the calculating of the immersion level may include: extracting eye-blink information, information on a change in outer corners of eyes and information on a change in corners of mouth from the face-related image; calculating an immersion-level score on the basis of the eye-blink information; generating emotion information of the user on the basis of the information on the change in outer corners of eyes and the information on the change in corners of mouth; and correcting the immersion-level score on the basis of the emotion information.

In addition, the correcting of the immersion-level score may include increasing the immersion-level score calculated on the basis of the eye-blink information by a specified value when the emotion information indicates an interested emotional state; or lowering the immersion-level score calculated on the basis of the eye-blink information by the specified value when the emotion information indicates an expressionless state.

A service device according to the exemplary embodiment of the present invention includes: a server communication circuit; and a server processor functionally connected to the server communication circuit. The server processor may be set to establish a communication channel with a terminal device by using the server communication circuit, provide specified content to the terminal device in response to a request of the terminal device, receive immersion-level information of a user related to the specified content from the terminal device, generate a content recommendation list on the basis of the immersion-level information, and transmit the content recommendation list to the terminal device.

In particular, the server processor may be set such that the higher the immersion-level score is, the higher a user rating for the content is, and the lower the immersion-level score is, the lower the user rating for the content is.

Here, the server processor may be set to search for other users who have given a same user rating for the content, search for the content in which ratings respectively given by the other users are greater than or equal to a specified value, and generate the content recommendation list on the basis of the searched content.

Here, the server processor may be set to search for other users who have given a same user rating for the content, search for the content in which ratings respectively given by the other users are greater than or equal to a specified value, and generate the content recommendation list on the basis of the searched content.

### Advantageous Effects

According to the present invention, the present invention supports the functionality in which an immersion level for content that the user is viewing may be more accurately calculated, the user's preference or rating for the content is determined on the basis of the accurately calculated immersion level, and more suitable recommended content may be suggested to the user on the basis of the immersion level.

In addition, various effects other than the above-described effects may be directly or implicitly disclosed in the detailed description according to an exemplary embodiment of the present invention to be described later.

### DESCRIPTION OF DRAWINGS

FIG. 1 is a view showing an example of a system environment related to managing immersion level according to an exemplary embodiment of the present invention.

FIG. 2 is a view showing an example of a configuration of a service device according to the exemplary embodiment of the present invention.

FIG. 3 is a view showing an example of a configuration of a terminal device according to the exemplary embodiment of the present invention.

FIG. 4 is a view showing an example of a configuration of a processor of the terminal device according to the exemplary embodiment of the present invention.

FIG. 5 is a view showing an example of a method for managing an immersion level according to the exemplary embodiment of the present invention.

### DESCRIPTION OF THE REFERENCE NUMERALS IN THE DRAWINGS

50: network

100: terminal device

110: communication circuit

120: input part

130: audio processing part

140: memory

150: processor

160: display

170: camera

200: service device

210: server communication circuit

240: server memory

250: server processor

### BEST MODE

In order to clarify the features and advantages of the problem solution of the present invention, the present invention will be described in more detail with reference to specific exemplary embodiments of the present invention shown in the accompanying drawings.

However, in the following description and the accompanying drawings, detailed descriptions of known functions or configurations that may obscure the subject matter of the present invention will be omitted. In addition, it should be noted that the same components are denoted by the same reference numerals as much as possible throughout the drawings.

The terms or words used in the following description and drawings should not be construed as being limited to their ordinary or dictionary meanings, and should be interpreted as meanings and concepts corresponding to the technical idea of the present invention based on the principle that the inventors may properly define the concept of the terms in order to best describe their invention. Therefore, the exemplary embodiments described in the present specification and the configurations shown in the drawings are only the most preferred exemplary embodiments of the present invention, and do not represent all the technical ideas of the present invention, and accordingly, it should be appreciated that there may be equivalents and modifications at the time when the present application is filed.

In addition, terms including ordinal numbers such as “first”, “second”, etc. are used to describe various components, and the terms are used only for the purpose of distinguishing one component from other components, and are not used to limit the above components. For example, a first component may be referred to as a second component without departing from the scope of the present invention, and similarly, the second component may be referred to as the first component.

In addition, the terms used herein are for the purpose of describing particular exemplary embodiments only and are not intended to be limiting the present invention. As used herein, the singular forms are intended to include the plural forms as well, unless the context clearly indicates otherwise. It will be further understood that the terms “comprise”, “include”, “have”, and the like, when used in the present specification, specify the presence of stated features, integers, steps, operations, elements, components, and/or combinations of them but do not preclude the presence or addition of one or more other features, integers, steps, operations, elements, components, and/or combinations thereof.

In addition, the terms “part”, “unit”, “module”, and the like mean a unit for processing at least one function or operation and may be implemented by a combination of hardware and/or software. In addition, unless otherwise indicated herein or clearly contradicted by context in the context of describing the present invention (especially in the context of the following claims), “a or an”, “one”, “the” and similar related words may be used in the meanings including both the singular and the plural.

In addition to the above terms, specific terms used in the following description are provided to aid understanding of the present invention, and the use of these specific terms may be changed to other forms without departing from the technical scope and spirit of the present invention.

In addition, the exemplary embodiments within the scope of the present invention include computer-readable media having or carrying computer-executable instructions or data structures stored in the computer-readable media. Such computer-readable media may be any available media accessible by a general purpose or special purpose computer system. As an example, such computer readable media may include RAM, ROM, EPROM, CD-ROM or other optical disk storage, and magnetic disk storage or other magnetic storage, or may include physical storage media such as any other media that can be used to store or deliver a predetermined program code means in the form of computer-executable instructions, computer-readable instructions, or data structures and accessed by a general purpose or special purpose computer system, but is not limited thereto.

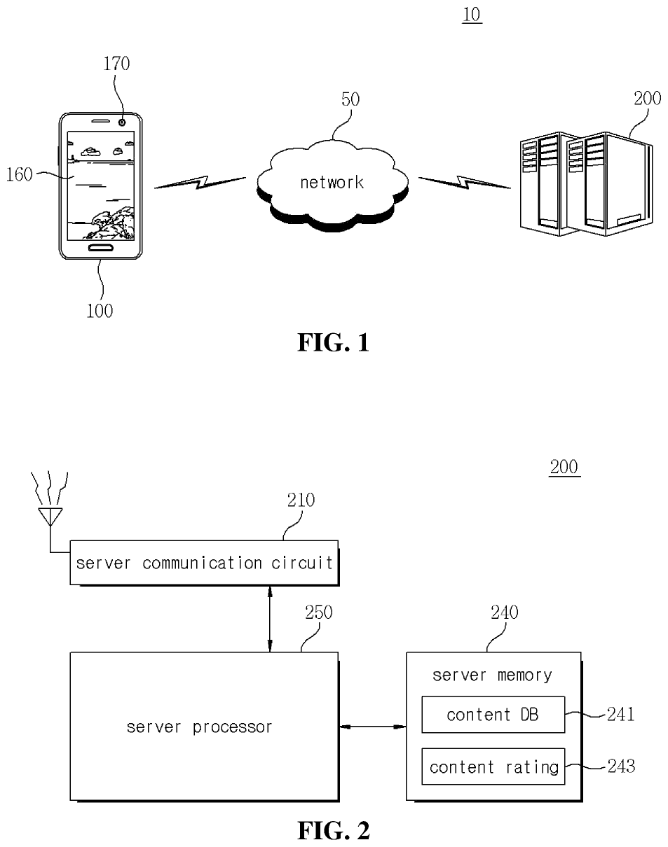

FIG. 1 is a view showing an example of a system environment related to managing an immersion level according to an exemplary embodiment of the present invention.

Referring to FIG. 1, a system environment 10 related to managing an immersion level according to the exemplary embodiment of the present invention may include: a network 50, a terminal device 100, and a service device 200.

In the system environment 10 related to managing an immersion level of the present invention described above, when the terminal device 100 accesses the service device 200 through the network 50, the service device 200 may provide specified content to the terminal device 100. The terminal device 100 outputs the content to a display 160, collects face-related information of a user by using a camera 170 while the content is being viewed, and then may calculate an immersion level for the corresponding content on the basis of the collected face-related information. The immersion level calculated by the terminal device 100 is provided to the service device 200, the service device 200 automatically assigns a user rating for the corresponding content on the basis of the immersion level, and a content recommendation list may be generated on the basis of the user rating and provided to the terminal device 100. In this operation, based on the rating given by the immersion level provided by the terminal device 100, the service device 200 may search for other users who gave the corresponding content the same or similar rating, search for content to which the other users have given the rating greater than or equal to a specified value, and provide a content recommendation list on the basis of the searched content.

The network 50 may be provided with not only an IP-based wired communication network such as an Internet network, but also a long-term evolution (LTE) network, a mobile communication network such as a WCDMA network, and various types of wireless networks such as a Wi-Fi network, and combinations thereof That is, the network environment related to a content recommendation function according to the present invention may be applied to all wired/wireless communication networks without distinction. Specifically, the network 50 may establish a communication channel between the service device 200 and the terminal device 100. For example, the network 50 may support at least one of 3G, 4G, and 5G wireless mobile communication methods that the service device 200 or the terminal device 100 may operate. Alternatively, the network 50 may establish a communication channel between the terminal device 100 and the service device 200 on a wired network basis. Such a network 50 should be interpreted as a concept including various wired networks, wireless networks, and networks of combination thereof that are currently developed and commercialized or will be developed and commercialized in the future.

The terminal device 100 may access the service device 200 through the network 50. Through a virtual page provided by the service device 200, the terminal device 100 may use at least one of a content search function, a content evaluation function, a content purchase function, and a content recommendation function, which are provided by the service device 200. The terminal device 100 may receive and output the content from the service device 200 for viewing the content. The terminal device 100 may obtain face-related information of a user while content is being output, and calculate an immersion level on the basis of the obtained face-related information.

Meanwhile, the terminal device 100 of the present invention may access the service device 200 according to a user input and perform a log-in operation, which is based on an ID and log-in information, on the service device 200. In this regard, the terminal device 100 may perform a procedure of accessing the service device 200 and pre-registering the login information. The terminal device 100 may provide an immersion level to the service device 200 in connection with a user rating input. The terminal device 100 may receive and output a content recommendation list generated by the service device 200 on the basis of the immersion level.

The service device 200 may have a communication standby state so that the terminal device 100 may access the service device 200 through the network 50. When accessed by the terminal device 100, the service device 200 may provide the terminal device 100 with a virtual page for using a content-related service provided by the service device 200 to the terminal device 100. The service device 200 may support tasks for the terminal device 100 to search for, purchase, or present content through the virtual page. In particular, the service device 200 may provide specified content to the terminal device 100 according to a request of the terminal device 100 and receive the immersion-level information for the corresponding content from the terminal device 100. The service device 200 may generate a content recommendation list on the basis of the immersion-level information and provide the generated content recommendation list to the terminal device 100. In this regard, the service device 200 may include a configuration as shown in FIG. 2.

FIG. 2 is a view showing an example of a configuration of a service device according to the exemplary embodiment of the present invention.

Referring to FIG. 2, the service device 200 of the present invention may include a server communication circuit 210, a server memory 240, and a server processor 250. The service device 200 of the present invention may construct at least a part of a server that supports a function of collecting immersion levels and recommending content according to the immersion levels. The server that supports the function of recommending the content may support searching for, sales, and the like of the content. In this regard, the server may be configured to provide sales or rental of real books, and may also provide sales or rental of electronic books.

The server communication circuit 210 may serve a role for establishing a communication channel of the service device 200. For example, the server communication circuit 210 may establish the communication channel with the terminal device 100 in response to an access request of the terminal device 100. The server communication circuit 210 may provide a specified virtual page (e.g., a content-related service page) to the terminal device 100 in response to control of the server processor 250. From the terminal device 100, the server communication circuit 210 may receive various messages (e.g., an input message, which is input by an input part of the terminal device 100, for requesting service use such as searching for, purchasing, and presenting content, or browsing at least a part of the content) related to the use of the virtual page. When receiving the various messages from the terminal device 100, the server communication circuit 210 may provide response messages (e.g. content recommendation list, content list, content data, etc.) respectively corresponding to the various messages in response to the control of the server processor 250. The server communication circuit 210 may receive the immersion-level information from the terminal device 100 and provide a content recommendation list generated on the basis of the immersion level to the terminal device 100.

The server memory 240 may store various data, programs, algorithms, and the like, which are related to operation of the service device 200. For example, the server memory 240 may include a content DB 241 and a content rating 243.

The content DB 241 may store at least one content. The content may include information on various content including video content such as movies and dramas, and text content such as novels or web novels, poetry, and essays. At least one content stored in the content DB 241 may be provided to the terminal device 100 according to a request from the terminal device 100. In addition, at least one content stored in the content DB 241 may be used to generate a content recommendation list.

For specific content, the content rating 243 may include at least one user rating input by users of the terminal device 100. Alternatively, the content rating 243 may include rating information for the specific content, the rating information being automatically assigned on the basis of the immersion level received from the terminal device 100. The content rating 243 may be classified and stored for each user. For example, the content rating 243 may store a rating assigned to content A on the basis of a level of immersion that a first user has immersed in the corresponding content A. In addition, the content rating 243 may store information of rating to be given by a second user on a plurality of content. The content rating 243 may be used to generate a content recommendation list.

The server processor 250 may perform tasks such as data transmission or data processing, which are related to operation of the service device 200. For example, the server processor 250 may perform tasks such as accessing the terminal device 100, providing a virtual page, and the like in relation to the content recommendation function. The server processor 250 may provide at least one content stored in the content DB 241 to the terminal device 100 according to a request of the terminal device 100. The server processor 250 may receive the immersion-level information of a user about the content provided to the terminal device 100. The server processor 250 may provide the rating information of the user for the corresponding content on the basis of the received immersion-level information. For example, the server processor 250 may give a high user rating depending on a magnitude of immersion level, and may give a lower user rating as the magnitude of immersion level decreases.

On the basis of the assigned rating, the server processor 250 may check other users who have given the same rating as that of the corresponding user from the content rating 243. In the content rating 243, the server processor 250 may detect content that has been given a rating greater than or equal to a specified value among ratings assigned by other users. The server processor 250 may generate a content recommendation list on the basis of the detected information. The server processor 250 may provide the generated content recommendation list to the terminal device 100. In this process, the server processor 250 may provide the content recommendation list to the terminal device 100 at a point in time when an advertisement is displayed during content playback on the terminal device 100. Alternatively, the server processor 250 may provide the content recommendation list in an advertisement display area on a content-playback screen of the terminal device 100. Alternatively, the server processor 250 may control the terminal device 100 to output the content recommendation list by using a pop-up window. Alternatively, the server processor 250 may control the display 160 to output the content recommendation list in an area where the viewing disturbance of the content-playback screen being played is minimal, for example, in a corner area among areas of the display 160 of the terminal device 100.

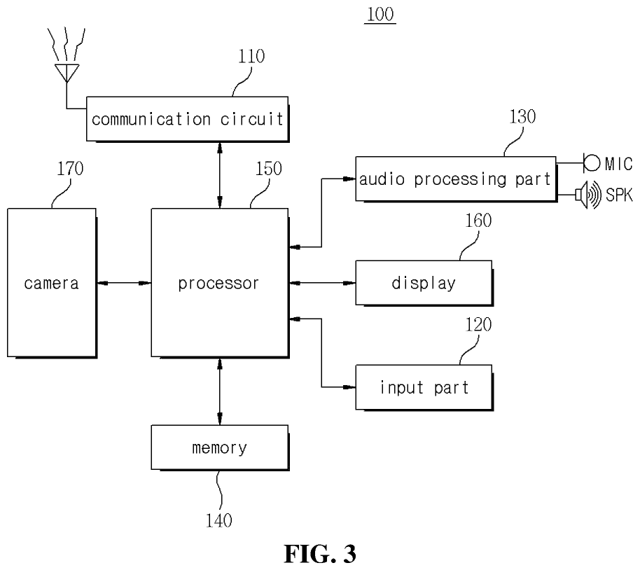

FIG. 3 is a view showing an example of a configuration of a terminal device according to the exemplary embodiment of the present invention.

Referring to FIG. 3, a terminal device 100 according to the exemplary embodiment of the present invention may include: a communication circuit 110, an input part 120, an audio processing part 130, a memory 140, a display 160, and a camera 170, and a processor 150.

The communication circuit 110 may support a communication function of the terminal device 100. For example, the communication circuit 110 may include a short-range communication circuit or a long-distance communication circuit. Alternatively, the communication circuit 110 may establish a communication channel with the service device 200 or other terminal devices, which provide content on the basis of a mobile communication base station.

Alternatively, the communication circuit 110 may receive the content provided by the service device through an Internet communication network or a broadcasting network. The communication circuit 110 may provide the received content to at least one of the displays 160 and the audio processing part 130 in response to the control of the processor 150. The communication circuit 110 may provide the immersion-level information to the service device 200 in response to the control of the processor 150 and receive the content recommendation list from the service device 200.

The input part 120 may generate a user input related to operation of the terminal device 100 and transmit the generated user input to the processor 150. For example, the input part 120 may include at least one component such as physical button or a keyboard, a touch pad, a touch screen, and an electronic pen. In response to user control, the input part 120 may generate the user input such as a user input related to a request for access to the service device 200, and a user input related to a selection of a specific content among at least one content provided by the service device 200.

The audio processing part 130 may perform audio processing of the terminal device 100, and may output or collect an audio signal according to the audio processing. In this regard, the audio processing part 130 may include a microphone MIC, a speaker SPK, etc. For example, the audio processing part 130 may output audio information among content received from the service device 200.

The memory 140 may store at least one of programs related to operation of the terminal device 100, data related to program operation, etc. The memory 140 may temporarily or semi-permanently store the content received from the service device 200. The memory 140 may store immersion-level information, and may store a content recommendation list received from the service device 200. In addition, the memory 140 may store at least one content.

The display 160 may output at least one screen related to the operation of the terminal device 100. For example, the display 160 may output a virtual page provided by the service device 200, a content-playback screen provided by the service device 200, and a content recommendation list provided by the service device 200. In addition, while viewing a specific content or after viewing of the content is completed, the display 160 may output immersion-level information about the corresponding content.

The camera 170 may collect an image of a subject. The camera 170 may be activated during content playback in response to the control of the processor 150 and may collect an image related to a user's face. The operation of the camera 170 may be ended together when the content playback is ended.

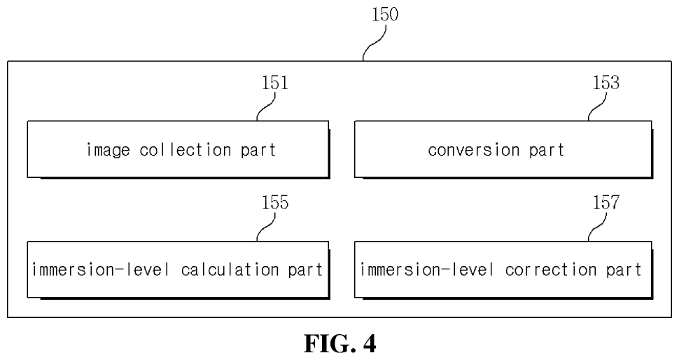

The processor 150 may access the service device 200 in response to a user input, receive content selected by the user from the service device 200, and output the content to the display 160. Alternatively, the processor 150 may select at least one content stored in the memory 140 according to the user input and output a playback screen of the selected content to the display 160. When the content is played back, the processor 150 may activate the camera 170 according to a preset setting and control the camera 170 to obtain a face-related image of a user. The processor 150 may calculate an immersion level on the basis of the obtained face-related image, and may map the calculated immersion level and the corresponding content to each other and store them in the memory 140. Alternatively, the processor 150 may provide the calculated immersion level to the service device 200. The processor 150 may include a configuration as shown in FIG. 4.

FIG. 4 is a view showing an example of a configuration of a processor of the terminal device according to the exemplary embodiment of the present invention.

Referring to FIG. 4, the processor 150 of the terminal device 100 according to the exemplary embodiment of the present invention may include: an image collection part 151, a conversion part 153, an immersion-level calculation part 155, and an immersion-level correction part 157.

The image collection part 151 may activate the camera 170 according to a setting. For example, when set to calculate an immersion level during a content playback, the image collection part 151 may automatically activate the camera 170 when a request to play content occurs. The image collection part 151 may control the camera 170 to collect an image of a user's face-related area. The image collection part 151 may transmit the collected images to the conversion part 153.

On the basis of the images transmitted from the image collection part 151, the conversion part 153 may collect information including: eye-blink information, information on a change in outer corners of eyes, information on a change in corners of mouth, etc.

In order to generate the eye-blink information, the conversion part 153 may extract an eye area from among the face area and detect a change in the image of the extracted eye area. For example, the conversion part 153 may first extract a face-related area from the received image by using a designated image processing technique, and then detect the eye area by applying the designated image processing technique to the extracted face-related area. On the basis of the change in the detected eye area, the conversion part 153 may determine whether the user's eye blinks or not. That is, the conversion part 153 may check contrast values of the eye area when the user closes his/her eyes and when the user opens his/her eyes. In this case, a determination part may determine whether the user's eye blinks by using the checked contrast values. The conversion part 153 may convert the data of whether the determined user's eyes blink into data of a discrete time series in the form of time information.

In this regard, the conversion part 153 may synchronize whether the user's eyes blink with a time when the corresponding user uses the content. The immersion-level calculation part 155 may analyze the immersion level of the user's content by using the converted data of the discrete time series. The immersion level may be determined by using a coefficient of variation (CV) of the user's eye blink interval, the coefficient of variation being identified in the data of the discrete time series. In the content with high immersion level, relatively few user's eye blink occurs, and thus the user's eye blink interval may be lengthened and the coefficient of variation may be increased. That is, the immersion-level calculation part 155 may determine that the higher the coefficient of variation of the user's eye blink interval, the higher the user's concentration on the content. Meanwhile, in the content with low concentration, the user's eye blink occurs relatively more, so that the user's eye blink interval may be shortened and the coefficient of variation may be decreased. The immersion-level calculation part 155 may determine that the lower the coefficient of variation of the user's eye blink interval, the lower the user's concentration of the content.

Meanwhile, the immersion-level calculation part 155 may reflect the characteristics of each user with respect to the number of eye blinks. In this regard, the immersion-level calculation part 155 may obtain an average value of the user's eye blink and calculate an immersion level on the basis of the average value. Alternatively, the immersion-level calculation part 155 may obtain an overall average value of eye blink of other users and calculate an immersion level according to a degree of eye blink of a user on the basis of the obtained overall average value.

The immersion-level correction part 157 may extract a value of a change in outer corners of eyes and a value of a change in corners of mouth from a face-related image and calculate emotion information on the basis of the extracted values. For example, on the basis of the value of a change in outer corners of eyes and value of a change in corners of mouth, the immersion-level correction part 157 may determine whether the user is currently in an interested emotional state. Alternatively, the immersion-level correction part 157 may determine whether the user is currently in a joyful state or an angry state by using the value of a change in outer corners of eyes and value of a change in corners of mouth. In this regard, the terminal device 100 may store and operate a database, an algorithm, and the like, which are capable of determining a user's emotional state in response to the value of a change in outer corners of eyes and value of a change in corners of mouth. The immersion-level correction part 157 may correct the immersion level on the basis of the user's emotion information. For example, even when the number of eye blinks is more than a specified number of times, the immersion-level correction part 157 may increase the calculated immersion level regardless of the number of eye blinks when the current user's emotional state is the interested emotional state. In addition, even when the number of eye blinks is less than the specified number of times, the immersion-level correction part 157 may relatively lower the level of immersion when the current emotional state of the user is in an expressionless state.

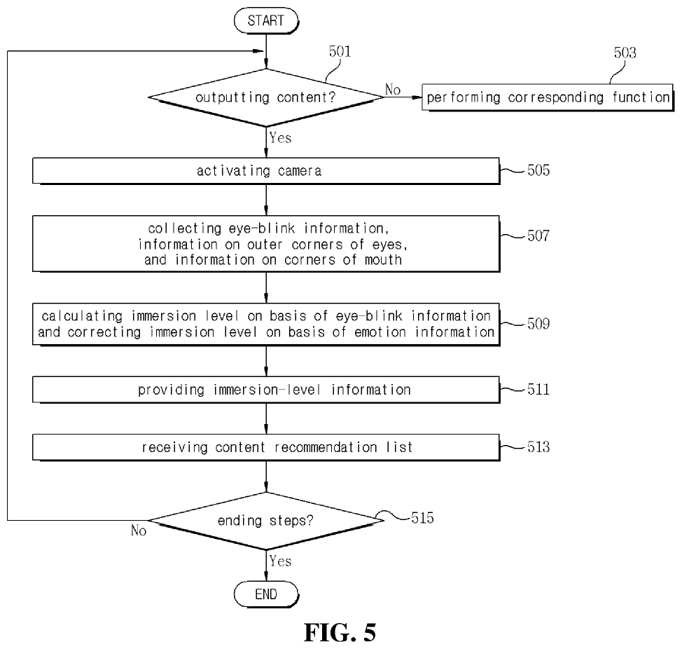

FIG. 5 is a view showing an example of the method for managing an immersion level according to the exemplary embodiment of the present invention.

Referring to FIG. 5, in relation to the method for managing an immersion level of the present invention, when an event related to the operation of the terminal device 100 occurs, the processor 150 of the terminal device 100 may check whether the event is related to outputting of content in step 502. When the generated event is an event that is not related to the outputting of the content, the processor 150 may perform a function corresponding to the event in step 503. For example, the processor 150 may support a call function or a music playback function in response to a type of event that has occurred.

When the generated event is an event related to the outputting of the content, the processor 150 may output the specified content on the display 160, and in step 505, activate the camera 170. In this process, at the start time of content playback, through the display 160, the processor 150 may inform the user that the immersion level is being calculated.

In step 507, by using the activated camera 170, the processor 150 may collect information related to the user's face, that is, information such as eye blink, movement of outer corners of eyes, and movement of corners of mouth. In this regard, the processor 150 may detect a face area by applying a first image processing method designated to the image obtained by the camera 170, and may detect an eye area, an area around the mouth, and the like, by applying a second image processing method designated to the face area. The first image processing method may include a method of extracting feature points from an image and detecting the face area by comparing the extracted feature points with predefined facial feature points. The second image processing method may include a method of extracting feature points from the face area, and detecting the eye area and the area around the mouth by comparing the extracted feature points with predefined distribution values of the feature points of the eye area and the feature points of the area around the mouth.

In step 509, the processor 150 may calculate the immersion level based on the eye-blink information. In addition, the processor 150 may perform immersion-level correction based on emotion information. For example, the processor 150 calculates an immersion-level score according to the number of eye blinks, wherein the lower the number of eye blinks, the higher the immersion-level score, and the higher the number of eye blinks, the lower the immersion-level score. When the immersion-level score is calculated, the processor 150 may correct the immersion level score calculated on the basis of the emotion information. For example, through information on a change in outer corners of eyes and information on a change in corners of mouth, the processor 150 may increase the immersion-level score when a current user is in an interested emotional state. Alternatively, the processor 150 may lower the immersion-level score when the current user is in an expressionless state identified through the information on a change in outer corners of eyes, information on a change in corners of mouth.

In step 511, the processor 150 may provide immersion-level information to the service device 200. The processor 150 may provide the immersion-level information to the service device 200 that has provided the content, or may provide the immersion-level information to the service device 200 that provides the specified content.

In step 513, the processor 150 may receive a content recommendation list from the service device 200. The processor 150 may output the received content recommendation list on the display 160.

In step 515, the processor 150 may check whether an event related to termination of a function of the terminal device 100 occurs. When the event related to the termination of the function occurs, the processor 150 may terminate the function of the terminal device 100. For example, the processor 150 may terminate a connection with the service device 200. Alternatively, the processor 150 may stop a playback of the content being output and turn off the display 160. When there is no event related to the termination of the function, the processor 150 may branch to the previous step of step 501 and perform the following operation again.

As described above, although the present specification includes the details of a number of specific exemplary embodiments, these should not be construed as limiting to the scope of any invention or claim that is available. Rather, it should be understood as a description of features that may be peculiar to a particular exemplary embodiment of a particular invention.

In addition, although the drawings are illustrated in a specific order, it should not be understood that such operations must be performed in that illustrated particular order or sequential order, or that all illustrated operations must be performed in order to obtain a desired result. In a certain case, multitasking and parallel processing may be advantageous. In addition, the separation of the various system components of the above-described embodiments should not be understood as requiring such separation in all embodiments, and it should be understood that the above-described program components and systems may generally be integrated together into a single software product or packaged into multiple software products.

The present description presents the best mode of the present invention, and provides examples for describing the present invention and for enabling those skilled in the art to make and use the present invention. The specification written in this way is not intended to limit the present invention to the specific terms presented. Accordingly, although the present invention has been described in detail with reference to the above-described examples, those skilled in the art can make modifications, changes, and deformation to the present examples without departing from the scope of the present invention.

Accordingly, the scope of the present invention should not be determined by the described exemplary embodiments, but should be determined by the claims.

According to the above-described method for managing an immersion level and an electronic device supporting the same of the present invention, the present invention may improve reliability of the user's preference for specific content by more accurately calculating the user's immersion level for the content. Accordingly, in the present invention, by recommending more suitable content to the user, it is possible to provide an environment in which the user may concentrate more on the content of interest while saving time required for content searching.

### INDUSTRIAL APPLICABILITY

According to the above-described method for managing an immersion level and an electronic device supporting the same of the present invention, the present invention may improve reliability of the user's preference for specific content by more accurately calculating the user's immersion level for the content. Accordingly, in the present invention, by recommending more suitable content to the user, it is possible to provide an environment in which the user may concentrate more on the content of interest while saving time required for content searching.

1. An electronic device comprising:
a display for outputting content;
a camera for collecting a user-related image; and
a processor functionally connected to the display and the camera,
wherein the processor is set to activate the camera while outputting a content-playback screen on the display when a playback of the content is requested, obtain a face-related image of a user by using the activated camera, extract eye-blink information, information on a change in outer corners of eyes, and information on a change in corners of mouth from the face-related image, and calculate an immersion level of the user with the content on the basis of the eye-blink information, the information on the change in outer corners of eyes, and the information on the change in corners of mouth.

2. The electronic device of claim 1, wherein the processor is set to calculate an immersion-level score on the basis of the eye-blink information, generate emotion information of the user on the basis of the information on the change in outer corners of eyes and the information on the change in corners of mouth, and correct the immersion-level score on the basis of the emotion information.

3. The electronic device of claim 2, wherein the processor is set to increase the immersion-level score calculated on the basis of the eye-blink information by a specified value when the emotion information indicates an interested emotional state.

4. The electronic device of claim 2, wherein the processor is set to lower the immersion-level score calculated on the basis of the eye-blink information by a specified value when the emotion information indicates an expressionless state.

5. The electronic device of claim 1, further comprising:
a communication circuit for establishing a communication channel with a service device for providing the content.

6. The electronic device of claim 5, wherein the processor is set to transmit an immersion-level score to the service device through the communication circuit, receive a content recommendation list generated on the basis of the immersion-level score from the service device, and output the received content recommendation list on the display.

7. A method for managing an immersion level, the method comprising:
receiving a playback request of content by a terminal device;
activating the camera while outputting a content-playback screen according to the playback request of the content;
obtaining a face-related image of a user by using the activated camera;
calculating the immersion level of the user for the content on the basis of the face-related image;
providing information of the calculated immersion level to a designated service device; and
outputting a content recommendation list by receiving the content recommendation list, which is generated on the basis of the information of the immersion level, from the service device.

8. The method of claim 7, wherein the calculating of the immersion level comprises:
extracting eye-blink information, information on a change in outer corners of eyes and information on a change in corners of mouth from the face-related image;
calculating an immersion-level score on the basis of the eye-blink information;
generating emotion information of the user on the basis of the information on the change in outer corners of eyes and the information on the change in corners of mouth; and
correcting the immersion-level score on the basis of the emotion information.

9. The method of claim 8, wherein the correcting of the immersion-level score comprises:
increasing the immersion-level score calculated on the basis of the eye-blink information by a specified value when the emotion information indicates an interested emotional state; or
lowering the immersion-level score calculated on the basis of the eye-blink information by the specified value when the emotion information indicates an expressionless state.

10. A service device comprising:
a server communication circuit; and
a server processor functionally connected to the server communication circuit,
wherein the server processor is set to establish a communication channel with a terminal device by using the server communication circuit, provide specified content to the terminal device in response to a request of the terminal device, receive immersion-level information of a user related to the specified content from the terminal device, generate a content recommendation list on the basis of the immersion-level information, and transmit the content recommendation list to the terminal device.

11. The service device of claim 10, wherein the server processor is set such that the higher the immersion-level score is, the higher a user rating for the content is, and the lower the immersion-level score is, the lower the user rating for the content is.

12. The service device of claim 11, wherein the server processor is set to search for other users who have given a same user rating for the content, search for the content in which ratings respectively given by the other users are greater than or equal to a specified value, and generate the content recommendation list on the basis of the searched content.
